(19) United States
(12) Patent Application Publication
(10) Pub. No.: US 2026/0338698 A1
(43) Pub. Date: Oct. 29, 2026

(54) AMBIENT TEMPERATURE SENSING FOR REAGENT CONTROL

(71) Applicant: Zodiac Pool Systems LLC, Carlsbad, CA (US)

(72) Inventors: Anthony Levacque, Carlsbad, CA (US); Timothy McNeil, Carlsbad, CA (US); Taylor Kuter, Carlsbad, CA (US)

(73) Assignee: Zodiac Pool Systems LLC, Carlsbad, CA (US)

(21) Appl. No.: 19/653,044

(22) Filed: Apr. 20, 2026

Related U.S. Application Data
(60) Provisional application No. 63/793,807, filed on Apr. 24, 2025.

(51) Int. Cl. G01N 21/77; G01N 33/18

(52) U.S. Cl. CPC G01N 21/77; G01N 33/18

(57) ABSTRACT

A system for monitoring water quality of a swimming pool or spa includes a reagent-based water sensing device, which may be configured to measure one or more water parameters using one or more liquid reagents. In some cases, the system includes an air temperature sensor, and the air temperature sensor may be configured to measure an air temperature within the reagent-based water sensing device. Additionally, or alternatively, the system may include a control system, and the control system may control usage of the one or more liquid reagents to measure the one or more water parameters based on an air temperature within the reagent-based water sensing device.

### REFERENCE TO RELATED APPLICATION

This application claims the benefit of and priority to U.S. Provisional Patent Application No. 63/793,807, filed on Apr. 24, 2025, and entitled AMBIENT TEMPERATURE SENSING FOR REAGENT CONTROL, the content of which is hereby incorporated by reference in its entirety.

### FIELD OF THE INVENTION

The invention relates to water testing or monitoring systems, and more particularly, but not necessarily exclusively, to water testing or monitoring systems for swimming pools or spas.

### BACKGROUND

Maintaining water quality is important for swimming pools, spas, hot tubs, and other water containing vessels (hereinafter “swimming pools or spas”) to avoid issues for users of the swimming pool or spa as well as equipment of the swimming pool or spa. For example, if the water chemistry of the swimming pool or spa is off, a health hazard may be posed to users and/or operation of various pool equipment and/or systems may be compromised.

Conventional approaches for monitoring water quality include test strips, liquid or reagent test kits, or digital testers, remote testing, and maintenance service calls. While test strips are generally user-friendly and inexpensive, test strips are generally the least accurate testing approach. Reagent testing is more accurate than test strips, but existing approaches may be complicated, are not user-friendly to pool owners, and do not account for reagent degradation over time and due to temperature, thereby leading to inaccurate readings and/or wasted resources. Digital testers are generally expensive and may drift and age over time, often requiring extensive maintenance for cleaning and calibration. Remote testing requires the labor-intensive process of taking a sample of water to a store or chemistry laboratory to analyze the sample, and may pose issues of sample contamination, improper storage, and/or a change in water chemistry during transit. Service calls rely on the availability of maintenance personnel and further rely on the expertise of the person to properly conduct water quality testing and to properly understand and interpret the results from such testing. As such, many existing approaches to monitoring water quality may be inaccurate, inconvenient, labor-intensive and time-consuming.

### SUMMARY

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various embodiments of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

According to certain embodiments, a system for monitoring water quality of a swimming pool or spa includes (i) a reagent-based water sensing device configured to measure one or more water parameters using one or more liquid reagents and (ii) an air temperature sensor configured to measure an air temperature within the reagent-based water sensing device.

According to some embodiments, a system for monitoring water quality of a swimming pool or spa includes (i) a reagent-based water sensing device configured to measure one or more water parameters using one or more liquid reagents and (ii) a control system configured to control usage of the one or more liquid reagents to measure the one or more water parameters based on an air temperature within the reagent-based water sensing device.

According to various embodiments, a method of monitoring a water parameter of a swimming pool or spa includes obtaining an air temperature within a reagent-based water sensing device configured to measure one or more water parameters using one or more liquid reagents and predicting a degradation of the one or more liquid reagents based on the measured air temperature.

In some embodiments, a non-transitory computer readable storage medium includes a plurality of instructions executable by one or more processors, the plurality of instructions comprising instructions which, when executed by the one or more data processors, cause the one or more data processors to perform actions including obtaining an air temperature within a reagent-based water sensing device configured to measure one or more water parameters using one or more liquid reagents and predicting a degradation of the one or more liquid reagents based on the measured air temperature.

Various implementations described in the present disclosure can include additional systems, methods, features, and advantages, which cannot necessarily be expressly disclosed herein but will be apparent to one of ordinary skill in the art upon examination of the following detailed description and accompanying drawings. It is intended that all such systems, methods, features, and advantages be included within the present disclosure and protected by the accompanying claims.

### BRIEF DESCRIPTION OF THE DRAWINGS

The features and components of the following figures are illustrated to emphasize the general principles of the present disclosure. Corresponding features and components throughout the figures can be designated by matching reference characters for the sake of consistency and clarity.

FIG. 1 illustrates a pool system with a water sensing device according to embodiments.

FIG. 2 illustrates the water sensing device of the pool system of FIG. 1 according to embodiments.

FIG. 3 illustrates a method of controlling the pool system of FIG. 1 according to embodiments.

### DETAILED DESCRIPTION

Described herein are systems and methods for monitoring water of a swimming pool or spa using a reagent-based water sensing device which measures one or more water parameters using one or more liquid reagents. In certain embodiments, the systems and methods described herein include an air temperature sensor for measuring an air temperature within the reagent-based water sensing device. The usage of the one or more liquid reagents to measure the one or more water parameters may be adjusted and/or controlled based on the measured air temperature. In some embodiments, the systems and methods described herein may predict a degradation of the one or more liquid reagents in the reagent-based water sensing device based on the measured air temperature. Additionally, or alternatively, the systems and methods described herein may identify a reagent correction parameter based on the measured air temperature and measure the one or more water parameters using the one or more liquid reagents and with the reagent correction parameter. The reagent correction parameter may include, but is not limited to, a volume of reagent used for a measurement, a type of reagent used for a measurement, a duration of a measurement, combinations thereof, and/or as otherwise desired.

Compared to traditional approaches, the systems and methods described herein may compensate for the degradation of reagents due to temperature degradation, thereby providing reliable and adaptive reagent-based measurements over an entire life of a reagent supply. The systems and methods described herein provide reagent-based testing which requires less frequent replacement of a reagent supply, thereby providing material and cost savings. Various other benefits and advantages may be realized with the systems, devices, and methods provided herein, and the aforementioned advantages should not be considered limiting.

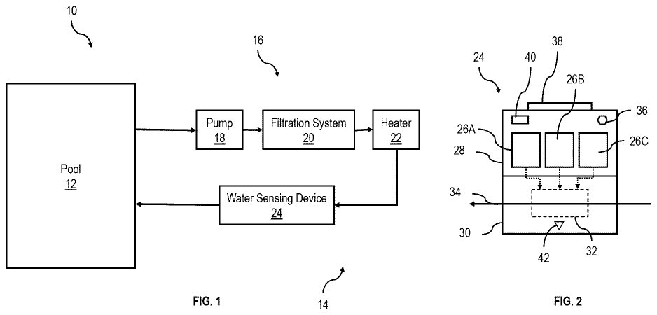

FIG. 1 illustrates an example of a pool system 10 according to embodiments. The pool system 10 generally includes a swimming pool or spa 12 (hereinafter “pool 12”) and a monitoring system 14 for measuring one or more water parameters of water of the pool 12 as described in greater detail below.

The pool 12 may have various features, arrangements, shapes, and profiles as desired, and the particular pool 12 illustrated should not be considered limiting. In some embodiments, the pool 12 includes a circulation system 16 for circulating water and/or performing various actions for controlling the water quality of the water of the pool 12. In certain embodiments, the circulation system 16 includes one or more pumps 18, a filtration system 20, and optionally a heater 22. In certain embodiments the circulation system 16 may include a sanitation system.

The monitoring system 14 may measure one or more water parameters such as but not limited to pH, free chlorine, total chlorine, total alkalinity, cyanuric acid concentration, and/or other parameters. In some embodiments, the monitoring system 14 may measure one water parameter, although in other embodiments, the monitoring system 14 may measure a plurality of water parameters, such as two water parameters, three water parameters, four water parameters, five water parameters, and/or more than five water parameters.

The monitoring system 14 may include one or more water sensing devices 24 for measuring the one or more water parameters of the water of the pool 12 using one or more reagents. As non-limiting examples, the one or more water sensing devices 24 may measure water parameters such as but not limited to pH, free chlorine, total chlorine, total alkalinity, cyanuric acid concentration, combinations thereof, and/or other parameters as desired.

While one water sensing device 24 is illustrated in FIG. 1, in other embodiments, any number of water sensing devices 24 may be utilized as desired. In some embodiments, a water sensing device 24 may be provided inline with one or more systems or equipment of the pool system 10. As an example, and as illustrated in FIG. 1, the water sensing device 24 may be provided inline with the water circulation system 16. In other embodiments, one or more water sensing devices 24 may be provided inline at other locations and/or relative to other equipment of the pool system 10 as desired. In other embodiments, one or more water sensing devices 24 need not be provided inline with one or more systems or equipment of the pool system 10. As non-limiting examples, one or more water sensing devices 24 may be provided and/or positioned regardless of other equipment and/or systems of the pool system 10, such as but not limited to as being provided as a hand held system, a floating system, combinations thereof, and/or as otherwise desired and suitable for measuring one or more water parameters.

In some embodiments, a single water sensing device 24 may measure any number of water parameters using reagents as desired, such as but not limited to one water parameter, two water parameters, three water parameters, four water parameters, five water parameters, and/or more than five water parameters.

In embodiments with a plurality of water sensing devices 24, the water sensing devices 24 may be a same type and/or configuration and/or may be different types and/or configurations as desired. As a non-limiting example, a plurality of water sensing devices 24 may include a first floating water sensing device 24 configured to measure pH and a second water sensing device 24 inline with the circulation system 16 for measuring pH, free chlorine, total chlorine, and total alkalinity. As another non-limiting example, a plurality of water sensing devices 24 may include a first water sensing device 24 inline with the circulation system 16 for measuring cyanuric acid concentration and a second water sensing device 24 inline with the circulation system 16 for measuring pH. In embodiments with a plurality of water sensing devices 24, the water sensing devices 24 may be various combinations of types of water sensing devices 24 as desired. In such embodiments, all the water sensing devices 24 need not be reagent-based water sensing devices, but at least one water sensing device 24 is a reagent-based water sensing device. Other combinations of water sensing devices 24 may be utilized as desired.

Referring to FIG. 2, the water sensing device 24 may include one or more stores of reagent 26. In certain embodiments, the reagent may be provided in the stores of reagent 26 in liquid form, although in other embodiments the reagent may be provided with the water sensing device 24 as desired. In FIG. 2, the water sensing device 24 includes three stores of reagent 26A-C. However, in other embodiments, the water sensing device 24 may include any number of stores of reagent 26 as desired. Optionally, the number of stores of liquid reagent 26 may at least partially depend on a number of water parameters that the water sensing device 24 is adapted to measure.

The stores of reagent 26 may have various constructions as desired. In some embodiments, the stores of reagent 26 may be constructed from a relatively flexible material such that the stores of reagent 26 have an adjustable shape and/or may at least partially conform to the volume of liquid reagent container therein. In one non-limiting example, the stores of reagent 26 are flexible pouches. In other embodiments, the stores of reagent 26 need not be constructed from a flexible material and instead may be constructed from a relatively rigid material which maintains its shape when empty.

In some embodiments, the stores of reagent 26 may be provided within a removable component 28 of the water sensing device 24, such as but not limited to a cartridge, a cover, a housing, combinations thereof, and/or other features as desired adapted to be selectively engaged with and/or disengaged from a base 30 of the water sensing device 24. In FIG. 2, the removable component 28 is a cartridge that selectively engages the base 30. The stores of reagent 26 within the removable component may allow for the supply of reagent to be replaced as needed without requiring a complete disassembly and/or removal of the water sensing device 24 from the pool system 10. In other embodiments, the stores of reagent 26 may be provided within the water sensing device 24 as desired and need not be within a removable component 28 (and the water sensing device 24 optionally may omit the removable component(s) 28).

In certain embodiments and as illustrated in FIG. 2, the water sensing device 24 includes a testing region 32. When the stores of reagent 26 are provided with the water sensing device 24, the stores of reagent 26 may be fluidly connected to the testing region 32 such that reagent from the stores of reagent 26 may be selectively provided (represented by dotted arrows) to the testing region 32. Within the testing region 32, the reagent may mix with the water (represented by arrow 34), and one or more water parameters of the water may be measured by one or more sensors 42. The one or more sensors 42 may be various suitable devices for measuring the mixed water and reagent and for obtaining a water parameter measurement. In one non-limiting example, the one or more sensors 42 may be light-based and/or optical-based sensors 42.

As illustrated in FIG. 2, in certain embodiments, the water sensing device 24 includes one or more air temperature sensors 36 for measuring an air temperature of the ambient air within the reagent-based water sensing device 24. While a single air temperature sensor 36 is illustrated in FIG. 2, in other embodiments, the water sensing device 24 may include any number of air temperature sensors 36 as desired, including a plurality of air temperature sensors 36. The air temperature sensor 36 may be provided at various locations within the water sensing device 24, including but not limited to the removable component 28 (if included), the base 30, and/or other locations or combinations thereof as desired. In some embodiments, the air temperature sensor 36 may be provided at a location suitable for measuring the air temperature of the portion of the water sensing device 24 housing the one or more stores of reagent 26. As described in greater detail below, the air temperature measurements from the air temperature sensor 36 may be utilized to predict a degradation of one or more reagents within the water sensing device 24, generate a reagent correction parameter, and/or provide a calibrated water parameter measurement to a user and/or other system of the pool system 10.

Optionally, the water sensing device 24 includes devices, components, and/or systems for receiving and/or providing data such as water parameter measurements to a user and/or another system. As non-limiting examples, the water sensing device 24 optionally may include a user interface 38 on or associated with the water sensing device 24. Additionally, or alternatively, the water sensing device 24 may include a communication module enabling wired or wireless communication between the water sensing device 24 and a remote device, such as but not limited to a handheld device, user device, phone, computer, tablet, combinations thereof, and/or other remote devices as desired via various communication techniques. In one non-limiting example, the water sensing device 24 may include a communication module enabling wireless communication between the water sensing device 24 and an application running on a user device, another system of the pool system 10 such as but not limited to sanitation system, and/or a control system of the pool system 10.

In various embodiments, in addition to the water sensing device 24, the monitoring system 14 may include a control system 40. In some embodiments, the control system 40 is a dedicated component of the monitoring system 14. In other embodiments, the control system 40 may be a control system of the pool system 10 and may be communicatively coupled to other systems in addition to the monitoring system 14.

The control system 40 may include one or more processing units and/or memory devices and is communicatively coupled to the components of the monitoring system 14. The control system 40 may be local to or remote from the monitoring system 14 and/or pool system 10 as desired. As non-limiting examples, in some embodiments, the control system 40 (or components thereof) may be remote from the water sensing device 24 and may be internet-based, in the cloud, at another physical location, combinations thereof, and/or as otherwise desired. Additionally, or alternatively, the control system 40 (or components thereof) may be provided locally within the monitoring system 14 and/or the pool system 10 as a stand-alone device and/or component of another device and/or system. As a non-limiting example, FIG. 2 illustrates the control system 40 as being onboard the water sensing device 24 in FIG. 2. As other non-limiting examples, one or more components of the control system 40 may be implemented as a stand-alone device, locally onboard a component of the circulation system 16, combinations thereof, and/or as otherwise desired. Other configurations of the control system 40 may be implemented as desired.

The one or more processing units of the control system 40 may be any suitable processing device or combinations of devices including but not limited to one or more application specific integrated circuits, digital signal processors, digital signal processing devices, programmable logic devices, field programmable gate arrays, processors, controllers, micro-controllers, microprocessors, other electronic units, and/or a combination thereof. The one or more memory devices of the control system 40 may be any machine-readable medium that can be accessed by the processor, including but not limited to any type of long term, short term, volatile, nonvolatile, or other storage medium, and is not to be limited to any particular type of memory or number of memories, or type of media upon which memory is stored. Moreover, as disclosed herein, the term “storage medium,” “storage” or “memory” can represent one or more memories for storing data, including read only memory, random access memory, magnetic random access memory, core memory, magnetic disk storage mediums, optical storage mediums, flash memory devices and/or other machine readable mediums for storing information. The term “machine-readable medium” includes, but is not limited to, portable or fixed storage devices, optical storage devices, wireless channels, and/or various other storage mediums capable of storing that contain or carry instruction(s) and/or data.

In various embodiments, the control system 40 may include one or more communication modules such that the control system 40 is communicatively coupled to at least the water sensing device 24 such that the control system 40 may obtain the air temperature measurements from the air temperature sensor 36. The control system 40 may be communicatively coupled to other components of the pool system 10 and/or systems or devices as desired such that the control system 40 may send and/or receive information with other components. As non-limiting examples, the control system 40 may be communicatively coupled to a user device. Non-limiting examples of communication modules may include systems and mechanisms enabling wired communication and/or wireless communication (e.g., near field, cellular, Wi-Fi, Bluetooth®, Bluetooth Low Energy (BLE), low-power wide area network, cloud-based communication etc.).

As mentioned, the control system 40 is communicatively coupled to at least the water sensing device 24 such that the air temperature measurements from the air temperature sensor 36 may be provided to the control system 40. In certain embodiments, reagents provided in the stores of reagent 26 may be sensitive to changes in ambient temperature and/or high ambient temperatures. As non-limiting examples, reagents used to measure pH, free chlorine, and alkalinity may be sensitive to high ambient temperatures and may degrade over time at such high ambient temperatures. The degradation of such reagents in turn compromises the reliability and accuracy of water parameter measurements made using such reagents, and users and/or equipment relying on such measurements in turn may incorrectly control and/or adjust the water chemistry of the pool 12, thereby potentially wasting resources and/or providing risks to both users of the pool and equipment of the pool. As a non-limiting example, Table 1 below provides stability test results for free chlorine reagents at 60 °C compared to a control in the range of 2-29 °C over six days. As illustrated in Table 1, the degradation of the free chlorine reagent at the elevated temperature over time resulted in a change in absorbance values compared to the control, which in turn caused changes in the free chlorine measurements over time (increasing the error both negatively and positively).

TABLE 1

| | RT control | | 60 °C | | |
|---|---|---|---|---|---|
| | abs | ppm | abs | ppm | error (ppm) |
| Day 1 | −0.008 | 0.0 | 0 | 0.0 | 0.0 |
| Day 2 | −0.005 | 0.0 | 0.009 | 0.0 | 0.1 |
| Day 3 | 0.323 | 1.2 | 0.279 | 1.0 | −0.2 |
| Day 4 | 0.321 | 1.1 | 0.28 | 1.0 | −0.1 |
| Day 5 | 1.789 | 6.5 | 1.499 | 5.5 | −1.0 |
| Day 6 | 1.698 | 6.2 | 1.452 | 5.3 | −0.9 |

In certain embodiments, and compared to traditional approaches, the control system 40 may provide reliable and accurate reagent-based water parameter measurements by monitoring the temperature within the water sensing device 24 and/or the ambient temperature at which the reagent is stored and predicting a degradation of the regents and/or compensating a water parameter measurement to account for the degradation of the reagents.

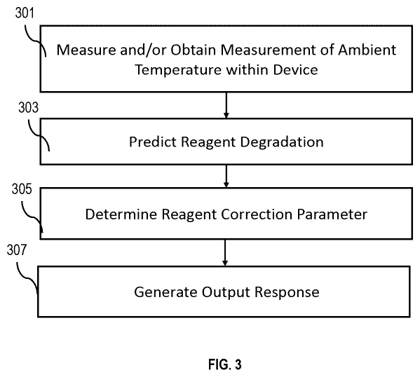

FIG. 3 illustrates an example of a method of monitoring one or more water parameters of the pool 12 and compensating for ambient temperature degradation of reagents. In some embodiments, the method and/or components may be performed by the control system 40. Other methods may be implemented using the monitoring system 14 to compensate for reagent degradation as desired.

In a block 301, the method includes measuring the ambient air temperature within the water sensing device 24 using the air temperature sensor 36 and providing the air temperature measurements to the control system 40. In some embodiments, the air temperature may be continuously measured by the air temperature sensor 36, although in other embodiments the air temperature sensor may measure the air temperature within the water sensing device 24 at various regular or irregular time intervals as desired.

In some embodiments, the method includes an optional block 303 in which the control system 40 predicts reagent degradation based on the temperature measurements obtained in block 301 and the period of time. In such embodiments, based on the reagent, the temperature or change in temperature, and the period of time, the control system 40 may predict the degradation of the reagent and/or a property of the reagent, optionally utilizing a predefined calibration curve. The predefined calibration curve may be defined by the control system 40 and/or provided to the control system 40 as desired. In certain embodiments, the control system 40 may determine and/or predict a degradation of the reagent based on ambient temperature and period of time at the temperature. As a non-limiting example and with reference back to Table 1, the control system 40 may predict a degradation of the free chlorine reagent based on the reagent being in an ambient temperature of 60 °C for one day, two days, three days, four days, five days, and/or six days. As another non-limiting example, the control system 40 may predict a degradation of the free chlorine reagent based on the current lifetime usage of the reagent and the monitored change in temperature of the lifetime usage of the reagent. Other calibration curves and/or predictions of reagent degradation may be implemented as desired.

In a block 305, the method includes determining, by the control system 40, a reagent correction parameter based on the temperature measurements obtained in block 301 and the time period. As a non-limiting example and with reference back to Table 1, based on an ambient temperature measurement of 60 °C over a time period of one day (e.g., the reagent was newly provided), the control system 40 may determine and/or estimate that the free chlorine measurement (ppm) will be substantially the same (or within an acceptable error or tolerance) as an expected or control. In this example, the control system 40 may determine that the reagent control parameter is 0 (e.g., any actual free chlorine measurement made at one day does not need to be corrected or adjusted). As a non-limiting example and with reference back to Table 1, based on an ambient temperature measurement of 60 °C over five days, the control system 40 may determine and/or estimate that the free chlorine measurement (ppm) will be off (in this example, less than) by 1.0 ppm due to the degradation of the reagent. In this example, the control system 40 may determine that the reagent correction parameter includes an adjustment of the measured free chlorine measurement by 1.0 ppm. Other reagent correction parameters may be identified in block 305 based on the particular reagent, the time period, and the ambient temperature measurements.

In a block 307, the method includes generating an output response based on the reagent correction parameter. In some embodiments, block 307 includes receiving a water parameter measurement (e.g., from sensor 42) and applying the reagent correction parameter to the water parameter measurement to generate a corrected measurement of the one or more water parameters. As a non-limiting example and with reference back to Table 1, block 307 may include obtaining and/or determining a free chlorine measurement of 5.5 ppm from the sensor 42 at day five, and applying the reagent correction parameter of 1.0 ppm to generate the corrected free chlorine measurement of 6.5 ppm. As a non-limiting example and with reference back to Table 1, block 307 may include obtaining and/or determining a free chlorine measurement of 0.0 ppm from the sensor 42 at day 1, and applying the reagent correction parameter of 0.0 ppm (e.g., no correction is made) to generate the corrected free chlorine measurement of 0.0 ppm.

Optionally, block 307 includes providing the corrected measurement to the user, such as via the user interface 38, a user device, an application on a user device, and/or as otherwise desired.

Optionally, block 307 includes providing the corrected measurement to one or more systems and optionally controlling such systems utilizing the corrected measurement. As a non-limiting example, based on the corrected measurement, the control system may automatically adjust and/or control a dispensing of chemicals into the pool (e.g., by controlling a sanitation/chemical dosing system and/or as otherwise desired).

Optionally, block 307 includes providing the reagent correction parameter, predicted degradation of a reagent, and/or other alert to a user and/or to another system without necessarily applying the reagent correction parameter to a current water parameter measurement. As non-limiting examples, the reagent correction parameter and/or predicted degradation of the reagent may be provided to a user to notify the user of the status of the reagent, notify the user of a change in reagent, prompt and/or suggest a replacement of the reagent, combinations thereof, and/or as otherwise desired.

Various other responses may be generated as desired, and the aforementioned examples should not be considered limiting.

In certain embodiments, blocks 301-307 may be performed over the use of the reagent such that the predicted reagent degradation and/or reagent correction parameter is adapted over use, thereby providing accurate and reliable water parameter measurements.

In various embodiments, the method includes resetting the reagent correction parameter when new reagent is supplied to the water sensing device 24. As a non-limiting example, the reagent correction parameter may be reset based on a detected and/or provided replacement of one or more reagents within the water sensing device 24.

Various other benefits and advantages may be realized with the systems, devices, and methods provided herein, and the aforementioned advantages should not be considered limiting.

Exemplary concepts or combinations of features of the invention may include:
A. A system for monitoring water quality of a swimming pool or spa, the system comprising (i) a reagent-based water sensing device configured to measure one or more water parameters using one or more liquid reagents and (ii) an air temperature sensor configured to measure an air temperature within the reagent-based water sensing device.
B. A system for monitoring water quality of a swimming pool or spa, the system comprising (i) a reagent-based water sensing device configured to measure one or more water parameters using one or more liquid reagents and (ii) a control system configured to control usage of the one or more liquid reagents to measure the one or more water parameters based on an air temperature within the reagent-based water sensing device.
C. A method of monitoring a water parameter of a swimming pool or spa, the method comprising:
   i. obtaining an air temperature within a reagent-based water sensing device configured to measure one or more water parameters using one or more liquid reagents; and
   ii. predicting a degradation of the one or more liquid reagents based on the measured air temperature.
D. A non-transitory computer readable storage medium comprising a plurality of instructions executable by one or more processors, the plurality of instructions comprising instructions which, when executed by the one or more data processors, cause the one or more data processors to perform actions including:
   i. obtaining an air temperature within a reagent-based water sensing device configured to measure one or more water parameters using one or more liquid reagents; and
   ii. predicting a degradation of the one or more liquid reagents based on the measured air temperature.
E. The system, method, or instructions of any preceding or subsequent statement or combination of statements, further comprising a control system configured to (i) receive the measured air temperature from the air temperature sensor and (ii) predict a degradation of the one or more liquid reagents based on the measured air temperature.
F. The system, method, or instructions of any preceding or subsequent statement or combination of statements, wherein the control system is further configured to determine a reagent correction parameter to measure the one or more water parameters using the one or more liquid reagents based on the predicted degradation.
G. The system, method, or instructions of any preceding or subsequent statement or combination of statements, wherein the reagent-water sensing device is configured to measure the one or more water parameters using the one or more liquid reagents and with the reagent correction parameter.
H. The system, method, or instructions of any preceding or subsequent statement or combination of statements, wherein the control system is further configured to determine a reagent correction parameter to measure the one or more water parameters using the one or more liquid reagents based on the predicted degradation.
I. The system, method, or instructions of any preceding or subsequent statement or combination of statements, wherein the control system is further configured to determine a reagent correction parameter based on the predicted degradation and apply the reagent correction parameter to a measurement of the one or more water parameters to generate a corrected measurement of the one or more water parameters.
J. The system, method, or instructions of any preceding or subsequent statement or combination of statements, wherein the control system is further configured to provide the corrected measurement of the one or more water parameters to a user via a user interface and/or communicating with a user device or system.
K. The system, method, or instructions of any preceding or subsequent statement or combination of statements, wherein the air temperature sensor is within the reagent-based water sensing device.
L. The system, method, or instructions of any preceding or subsequent statement or combination of statements, wherein the air temperature sensor is within a cartridge of the reagent-based water sensing device.
M. The system, method, or instructions of any preceding or subsequent statement or combination of statements, further comprising (i) detecting a replacement of the one or more liquid reagents and (ii) resetting the reagent correction parameter.
N. The system, method, or instructions of any preceding or subsequent statement or combination of statements, wherein the reagent-based water sensing device is configured to measure at least one of free chlorine, pH, ORP, total chlorine, total alkalinity, and/or cyanuric acid concentration using the one or more liquid reagents.

These examples are not intended to be mutually exclusive, exhaustive, or restrictive in any way, and the invention is not limited to these example embodiments but rather encompasses all possible modifications and variations within the scope of any claims ultimately drafted and issued in connection with the invention (and their equivalents). For avoidance of doubt, any combination of features not physically impossible or expressly identified as non-combinable herein may be within the scope of the invention. Further, although applicant has described devices and techniques for use principally with swimming pools or spas, persons skilled in the relevant field will recognize that the present invention conceivably could be employed in connection with other water containing vessels and in other manners, particularly but not limited to underwater installations. Finally, references to “pools” and “swimming pools” herein may also refer to spas or other water containing vessels used for recreation, training, or therapy.

As used herein, the meaning of “a,” “an,” and “the” includes singular and plural references unless the context clearly dictates otherwise.

The subject matter of embodiments of the present disclosure is described here with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described. A zero angle refers to an angle of 0° or 180°. Directional references such as “up,” “down,” “top,” “bottom,” “left,” “right,” “vertical,” “horizontal,” “lateral,” “longitudinal,” “front,” and “back,” among others, are intended to refer to the orientation as illustrated and described in the figure (or figures) to which the components and directions are referencing.

The terms “comprising,” “having,” “including,” and “containing” are to be construed as open-ended terms (i.e., meaning “including, but not limited to,”) unless otherwise noted. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

The above-described aspects are merely possible examples of implementations, merely set forth for a clear understanding of the principles of the present disclosure. Many variations and modifications can be made to the above-described embodiment(s) without departing substantially from the spirit and principles of the present disclosure. All such modifications and variations are intended to be included herein within the scope of the present disclosure, and all possible claims to individual aspects or combinations of elements or steps are intended to be supported by the present disclosure. Moreover, although specific terms are employed herein, as well as in the claims that follow, they are used only in a generic and descriptive sense, and not for the purposes of limiting the described embodiments, nor the claims that follow.

That which is claimed:

1. A system for monitoring water quality of a swimming pool or spa, the system comprising:
a reagent-based water sensing device configured to measure one or more water parameters using one or more liquid reagents; and
an air temperature sensor configured to measure an air temperature within the reagent-based water sensing device.

2. The system of claim 1, further comprising a control system configured to control usage of the one or more liquid reagents to measure the one or more water parameters based on the air temperature within the reagent-based water sensing device.

3. The system of claim 1, further comprising a control system configured to (i) receive the measured air temperature from the air temperature sensor and (ii) predict a degradation of the one or more liquid reagents based on the measured air temperature.

4. The system of claim 3, wherein the control system is further configured to determine a reagent correction parameter to measure the one or more water parameters using the one or more liquid reagents based on the predicted degradation.

5. The system of claim 4, wherein the reagent-water sensing device is configured to measure the one or more water parameters using the one or more liquid reagents and with the reagent correction parameter.

6. The system of claim 3, wherein the control system is further configured to:
determine a reagent correction parameter to measure the one or more water parameters using the one or more liquid reagents based on the predicted degradation;
detect a replacement of the one or more liquid reagents; and
reset the reagent correction parameter.

7. The system of claim 3, wherein the control system is further configured to determine a reagent correction parameter based on the predicted degradation and apply the reagent correction parameter to a measurement of the one or more water parameters to generate a corrected measurement of the one or more water parameters.

8. The system of claim 7, wherein the control system is further configured to provide the corrected measurement of the one or more water parameters to a user via a user interface and/or communicating with a user device or system.

9. The system of claim 1, wherein the air temperature sensor is within the reagent-based water sensing device.

10. The system of claim 1, wherein the air temperature sensor is within a cartridge of the reagent-based water sensing device.

11. The system of claim 1, wherein the reagent-based water sensing device is configured to measure at least one of free chlorine, pH, ORP, total chlorine, total alkalinity, and/or cyanuric acid concentration using the one or more liquid reagents.

12. A method of monitoring a water parameter of a swimming pool or spa, the method comprising:
obtaining an air temperature within a reagent-based water sensing device configured to measure one or more water parameters using one or more liquid reagents; and
predicting a degradation of the one or more liquid reagents based on the measured air temperature.

13. The method of claim 12, further comprising determining a reagent correction parameter to measure the one or more water parameters using the one or more liquid reagents based on the predicted degradation.

14. The method of claim 12, further comprising:
determining a reagent correction parameter based on the predicted degradation; and
applying the reagent correction parameter to a measurement of the one or more water parameters to generate a corrected measurement of the one or more water parameters.

15. The method of claim 14, further comprising providing the corrected measurement of the one or more water parameters to a user via a user interface and/or communicating with a user device or system.

16. The method of claim 14, further comprising (i) detecting a replacement of the one or more liquid reagents and (ii) resetting the reagent correction parameter.

17. A non-transitory computer readable storage medium comprising a plurality of instructions executable by one or more processors, the plurality of instructions comprising instructions which, when executed by the one or more data processors, cause the one or more data processors to perform actions including:
obtaining an air temperature within a reagent-based water sensing device configured to measure one or more water parameters using one or more liquid reagents; and
predicting a degradation of the one or more liquid reagents based on the measured air temperature.

18. The non-transitory computer readable storage medium of claim 17, wherein the instructions further comprise instructions causing the one or more data processors to perform actions including:
determining a reagent correction parameter to measure the one or more water parameters using the one or more liquid reagents based on the predicted degradation.

19. The non-transitory computer readable storage medium of claim 17, wherein the instructions further comprise instructions causing the one or more data processors to perform actions including:
determining a reagent correction parameter based on the predicted degradation; and
applying the reagent correction parameter to a measurement of the one or more water parameters to generate a corrected measurement of the one or more water parameters.

20. The non-transitory computer readable storage medium of claim 19, wherein the instructions further comprise instructions causing the one or more data processors to perform actions including:
(i) detecting a replacement of the one or more liquid reagents and (ii) resetting the reagent correction parameter.
